(19) United States
(12) Patent Application Publication
(10) Pub. No.: US 2004/0091528 A1
(43) Pub. Date: May 13, 2004

(54) Soluble drug extended release system

(75) Inventors: Victoria Rogers, San Bruno, CA (US); Philippe J.M. Dor, Cupertino, CA (US); Joseph A. Fix, Half Moon Bay, CA (US); Hiroyuki Kojima, Shizuoka (JP); Kazuhiro Sako, Shizuoka (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Yamanouchi Pharma Technologies, Inc., Palo Alto, CA; Yamanouchi Pharmaceutical Co. Ltd., Tokyo (JP)

(21) Appl. No.: 10/293,885

(22) Filed: Nov. 12, 2002

(51) Int. Cl. A61K 9/127; A61K 9/22

(52) U.S. Cl. 424/468; 424/450

(57) ABSTRACT

This invention relates to novel oral sustained-release formulations for delivery of an active agent (e.g., a drug), especially a highly water soluble drug. More particularly, this invention relates to novel formulations comprising a micelle-forming drug having a charge and at least one polymer having an opposite charge. Methods of using the novel formulations are also provided.

### CROSS-REFERENCE TO RELATED APPLICATIONS

This application claims priority to U.S. patent application Ser. No. 10/007,877, filed Nov. 13, 2001, converted to U.S. Provisional Application No. ______, incorporated herein by reference in its entirety for all proposes.

### FIELD OF THE INVENTION

This invention relates to novel oral sustained release formulations for delivery of an active agent (e.g., a drug), especially a highly water-soluble drug. More particularly, this invention relates to novel formulations comprising a micelle-forming drug having a charge and at least one polymer having an opposite charge.

### BACKGROUND OF THE INVENTION

Administration of drugs via conventional oral and intravenous methods severely limits the effectiveness of most drugs. Instead of maintaining drug levels within therapeutic windows, these methods cause an initial, rapid rise in plasma concentration levels followed by a rapid decline below therapeutic levels as the drugs are metabolized by the body. Therefore, repeated doses are necessary to maintain drugs at therapeutic levels for a sufficient period of time to achieve a therapeutic effect. To address this problem, numerous sustained release preparations have been developed to eliminate the initial burst effect and allow drug release at constant levels.

Polymeric formulations are typically used to achieve extended drug release (see, Langer et al. Nature 392:6679 supp. (1998)). Various successfull polymeric sustained release preparations have been developed for release of drugs with different physical properties. Such preparations have been extremely effective for increasing release times for relatively hydrophobic and water-insoluble drugs.

However, due to rapid drug diffusion through polymer matrices, it has been difficult to achieve extended release for highly soluble drugs using current sustained release technologies. Thus, there is a need for new formulations and processes which are capable of reducing drug diffusion and eliminating a burst effect of highly water-soluble drugs. The present invention fulfills these and other needs.

### BRIEF SUMMARY OF THE INVENTION

The present invention provides inter alia, an oral sustained release preparation comprising a micelle-forming drug and an oppositely charged polymer. Although a concept of a micelle is well known for the field of the surfactant or drug carrier, application of a micelle-forming drug to the sustained release formulation is not known at all. Furthermore it is really surprising that this formulation is excellent effective on the extended release of active agents, especially water-soluble drugs. A further advantage lies in the ability of the formulation to provide slow release even when the formulation contains large drug loads.

As such, the present invention provides an oral sustained release pharmaceutical formulation, comprising: a micelle forming drug having a charge; and at least one polymer having an opposite charge, further if necessary hydrogel-forming polymer substance and hydrophilic base. The micelle forming drug may have a positive charge or a negative charge at physiological pH.

In another embodiment, the present invention provides a method for modulating a micelle forming drug release profile, comprising varying the molar ratio of micelle forming drug having a charge with at least one polymer having an opposite charge, varying the additional amount of polymer having an opposite charge, thereby modulating the micelle forming drug release profile. Suitable micelle forming drugs include, for example, antidepressants, β-adrenoceptor blocking agents, anesthetics, antihistamines and the like. Preferably, the micelle forming drug is a water-soluble drug.

In another embodiment, the present invention provides a method for extending release of a micelle forming drug, comprising: orally administering a pharmaceutical formulation comprising a micelle forming drug having a charge; and at least one polymer having an opposite charge, thereby extending release of the micelle forming drug.

In another embodiment, the present invention provides a method for extending release of a micelle forming drug, comprising: orally administering a pharmaceutical formulation comprising a micelle forming drug having a charge; and at least one polymer having an opposite charge, further if necessary hydrogel-forming polymer substance and hydrophilic base, thereby extending release of the micelle forming drug.

Further objects and advantages will become more apparent when read with the drawings and detailed description, which follow.

### DEFINITIONS

The term “active agent” means any drug that can be carried in a physiologically acceptable tablet for oral administration. Preferred active agents include, micelle forming active agents capable of forming electrically charged colloidal particles.

The term “cps” or “centipoise” is a unit of viscosity=m Pascal second. The viscosity is measured by Broolfield or other viscometers. See, e.g., Wang et al. Clin. Hemorheol. Microcirc. 19:25-31 (1998); Wang et al. J. Biochem. Biophys. Methods 28:251-61 (1994); Cooke et al. J. Clin. Pathol. 41:1213-1216 (1998).

The term “carrageenan” as herein refers to all forms of a water-soluble extract from carrageenan, Irish moss, seaweed from the Atlantic coasts of Europe and North America. Sources include, e.g., Viscarin® 109 and Gelcarin®, such as GP-911, GP-812, GP-379, GP-109, GP-209 commercially available from FMC. Carageenans are high molecular weight, highly sulfated, linear molecules with a galactose backbone. They are made up of sulfated and nonsulfated repeating units of galactose and 3,6 anhydrogalactose, which are joined by alternating α-(1-3) and β-(1-4) glycosidic linkages. Another commercial source of carageenans is Sigma and Hercules Inc.

The term “polyacrylic acid” or “PAA” as used herein includes all forms and MWs of PAA polymers. Sources include, for example, Carbopol 971 from B.F. Goodrich.

The term “polyethylene oxide polymer” or “PEO” as used herein includes all forms and MWs of PEO polymers. Sources of PEO polymers include, e.g., Polyox WSR-303™ (average MW: 7×10⁶; viscosity 7500-10000 cps, 1% in H₂O, 25° C.); Polyox WSR Coagulant™ (average MW 5×10⁶; viscosity 5500-7500 cps, under the same conditions as above); Polyox WSR-301™ (average MW 4×10⁶; viscosity 1650-5500 cps, under the same conditions as above); Polyox WSR-N-60K™ (average MW 2×10⁶; viscosity: 2000-4000 cps, 2% in H₂O, 25° C.); all of which are trade names of Union Carbide Co. See also WO 94/06414, which is incorporated herein by reference.

The term “polyethylene glycol” or “PEG” as used herein includes all forms and MWs of PEG polymers. Sources of PEG polymers include Macrogol 400, Macrogol 1500, Macrogol 4000, Macrogol 6000, Macrogol 20000; all of which are trade names of Nippon Oil and Fats Co.

The terms “hydroxypropylmethylcellulose,” “sodium carboxymethylcellulose,” “hydroxyethylcellulose,” and “carboxyvinyl polymer” incorporate their common usages. Sources include: for hydroxypropylmethylcellulose (HPMC), e.g., Metolose 90SH100000™ (viscosity: 2900-3900 cps, under the same conditions as above); Metolose 90SH30000™ (viscosity: 25000-35000 cps, 2% in H₂O, 20° C.); all of which are trade names of Shin-Etsu Chemicals Co. For sodium carboxymethyl-cellulose (CMC-Na), e.g., Sanlose F-150MC™ (average MW 2×10⁵; viscosity 1200-1800 cps, 1% in H₂O, 25° C.), Sanlose F-1000MC™ (average MW 4.2×104; viscosity 8000-12000 cps, under the same conditions as above); Sanlose F-300MC™ (average MW 3×10⁵; viscosity 2500-3000 cps, under the same conditions as above), all of which are trade names of Nippon Seishi Co., Ltd. For hydroxyethylcellulose (HEC) (e.g., HEC Daicel SE850™), average MW 1.48×10⁶; viscosity: 2400-3000 cps, 1% in H₂O, 25° C.; HEC Daicel SE900™, average MW 1.56×10⁶; viscosity 4000-5000 cps, under the same conditions as above; all of which are trade names of Daicel Chemical Industries. For carboxyvinyl polymers, e.g., Carbopol 940™, average MW ca. 25×10⁵; B.F. Goodrich Chemical Co.

The term “therapeutic drug” as used herein means any drug that can be delivered in an orally delivered physiologically acceptable tablet.

The term “micelle forming” refers to any compound that is capable of forming electrically charged colloidal particles, ions consisting of oriented molecules, or aggregates of a number of compounds/molecules held loosely together by secondary bonds.

### BRIEF DESCRIPTION OF THE DRAWINGS

FIG. 1 illustrates soluble drug (10 wt. %) release from a 400 mg PAA/PEO matrix in Simulated Intestinal Fluid (SIF).

FIG. 2 illustrates the correlation between T₅₀ and log P for basic highly soluble drugs released from a 400 mg PAA/PEO (1:1.5) tablet.

FIG. 3 illustrates the correlation between critical micelle concentration (CMC) and log P.

FIG. 4 illustrates examples of charged drugs (either positive or negative) suitable for use in the release experiments.

FIG. 5 illustrates the release of negatively charged drugs from a PAA/PEO matrix.

FIG. 6 illustrates Diltiazem HCl release from PAA/polysaccharide matrix tablets (400 mg) in SGF (FIG. 6a) and SIF (FIG. 6b).

FIG. 7 illustrates Diltiazem HCl release from PAA/sulfated polymer matrix tablets (400 mg) in SGF (FIG. 7a) and SIF (FIG. 7b).

FIG. 8 illustrates Diltiazem HCl release from different matrix tablets in SGF (FIG. 8a) and SIF (FIG. 8b).

FIG. 9 illustrates Diltiazem HCl (25 wt. %) release from PAA/carrageenan (1:1) matrix in SGF and SIF.

FIG. 10 illustrates PAA/carrageenan ratio optimization for a formulation with 25 wt % Diltiazem HCl.

FIG. 11 illustrates release rates of Diltiazem HCl (60 wt. %) from matrix tablets with different PAA/carrageenan ratios in SGF (FIG. 11a) and SIF (FIG. 11b).

FIG. 12 illustrates Diltiazem HCl release from PAA/Viscarin 109 matrix at different drug loads in SGF (FIG. 12a) and SIF (FIG. 12b).

FIG. 13 illustrates Diltiazem HCl (25 wt. %) release from competitive systems based on carrageenan in SGF (FIG. 13a) and SIF (FIG. 13b).

FIG. 14 illustrates Diltiazem HCl (25 wt. %) release from competitive systems based on PAA in SGF.

FIG. 15 illustrates Diltiazem HCl (60 wt. %) release from competitive systems in SGF (FIG. 15a) and SIF (FIG. 15b).

FIG. 16 illustrates the effect of additional amount of PAA on Diltiazem HCl (50 wt. %) release in JP 2nd fluid.

FIG. 17 illustrates the effect of additional amount of PAA/carrageenan on Diltiazem HCl (50 wt. %) release in JP 2nd fluid.

### DETAILED DESCRIPTION OF PREFERRED EMBODIMENTS

This present invention provides, inter alia, an oral sustained release preparation comprising a micelle-forming active agent (i.e., drug) and an oppositely charged polymer forming a hydrogel matrix. The formulation is typically manufactured by direct compression of the drug and the polymeric excipient.

Advantageously, this formulation provides an extremely low release rate of active agent. In a preferred aspect, hydrogen-bonded complexes between the oppositely charged polymers and drug micelles prevent rapid diffusion of the drug. Without being bound by any particular theory, it is believed that drug release occurs when the charge of the polymer is neutralized by OH ions at the matrix/dissolution border and these bonds are disrupted.

In one embodiment, the number of administrations of the formulation can be reduced, thereby increasing patient compliance. Further, side effects of the drug can be reduced by suppressing rapid increases in blood concentration of the drug (seen in standard formulations). A further advantage of this formulation is that the release rates of the formulations are not significantly affected by loading with high amounts of drug.

Factors and events which form a theoretical basis for the embodiments of the invention are discussed herein. However, this discussion is not in any way to be considered as binding or limiting on the present invention. Those of skill in the art will understand that the various embodiments of the invention may be practiced regardless of the model used to describe the theoretical underpinnings of the invention.

I. Active Agents of the Invention

Active agents of this invention can be any drugs which form micelles. Micelle formation has been observed for antidepressants, β-adrenoceptor blocking agents, anesthetics, antihistamines, phenothiazines, antiacetylcholines, tranquilizers, antibacterials, and antibiotics (see, Attwood et al., J. Pharm. Pharmac., 30, 176-180 (1978); Attwood et al., J. Pharm. Pharmac., 31, 392-395 (1979); Attwood et al., J. Pharm. Pharmac., 38, 494-498 (1986); Attwood J. Pharm. Pharmac., 24, 751-752 (1972); Attwood et al. J. Pharm. Sci.v.63, no. 6, 988993 (1974); Attwood, J. Phar. Pharmacol., 28, 407-409 (1976)). Representative micelle-forming antidepressant drugs include imipramine HCl, omipramol HCl, and amitriptuline HCl. Representative micelle-forming β-adrenoceptor blocking agents include oxprenolol HCl, acebutolol HCl and solatol HCl. Representative micelle-forming anesthetics include procaine HCl, lidocaine HCl, and amethocaine HCl. Representative micelle-forming antihistamines include diphenhydramine HCl, chlorcyclizine HCl, diphenylpyraline HCl, promethazine HCl, bromodiphenhydramine HCl, tripelennamine HCl, and mepyramine maleate. Representative micelle-forming phenothiazines include chlorpromazine HCl, and promethazine HCl. Other micelle-forming drugs include tranquilizers, antibacterials and antibiotics.

In certain aspects, the active agents include, but are not limited to, betacaine hemisulphate, cinchocaine hydrochloride BP and lignocaine hydrochloride (Sigma); prilocaine hydrochloride BP bupivacaine hydrochloride (Astra Pharmaceuticals) mepivacaine hydrochloride (Leo) proparacaine hydrochloride (Squibb) and amethocaine hydrochloride BP (Smith and Nephew Pharmaceuticals). In certain other aspects, the following active ingredients are useful in the present invention. These include, but are not limited to, (4′-(1-hydroxy-2-Isopropyl-aminoethyl)methanesulphonanilfide) (Duncan, Flockhart); labetolol [5-(1-hydroxy-2-(1-methyl-3-phenyl-propylamino)ethyl) salicylamide] (Allen and Hanburys); aceburtolol ((±)-3′-acetyl-4′-(2-hydroxy-3-Isopropylaminopropoxy)-butyranilde) (May and Baker); propranolol {(±)-1-Isopropylamino-3-naphth-1′-yloxypropan-2-ol} (ICI) and oxprenolol {(±)-1-(o-allyloxyphenoxy)-3-Isopropylaminopropan-2-ol)} (Ciba); timolol maleate {(−)-1-butylamino-3(4-morpholino-1,2,5-thiediazol-3-yl-oxy)propan-2-ol maleate) (Merck, Sharp and Dohme); metroprolol lartrale ((±)-1-Isopropylamino 3-p-(2-methoxyethyl) phenoxypropan-2-ol tartrate} (Geigy Pharmaceuticals). In another embodiment, the active ingredients include, but are not limited to, adiphenine hydrochloride (Ciba); poldine methylsulphate B.P. (Beecham Research); lachesino chloride B.P.C. (Vestric); chlorphenoxamine hydrochloride (Evans Medical); piperiodolate hydrochloride and pipenzolate bromide (M.C.P. Pharmaceuticals); orphenadrine hydrochloride B.P. (Brocades, Gt Britain); benztropine mesylate B.P. (Merck Sharp and Dohme); clidinium bromide (Roche); ambutonium bromide (Wyeth) and benzilonitum bromide (Parke-Davis). Diphenhydramine hydrochloride B.P. (2-diphenylmothoxy-NN-dimethylethylamine hydrochloride) and chlorcyclizine hydrochloride B.P. [1-(p-chlorodiphenylmethyl)-4-methyl piperazine hydrochloride] obtained from Parke-Davis and Company and Burroughs Wellcome and Company respectively. Bromodiphenhydramine hydrochloride [2-(α-p-bromophenyl-α-phenylmethoxy)-NN-dimethylethylamine hydrochloride] and dipenylpyraline hydrochloride (4-diphenyl-methoxy-1-methylpiperidine hydrochloride) respectively. Those of skill in the art will know of other active ingredients suitable for use in the present invention.

In preferred embodiments, active agents of this invention are highly water soluble drugs. And further preferred embodiments, active agents of this invention are basic drugs. This invention is particularly useful for such drugs, which exhibit a strong burst effect due to rapid diffusion through polymeric matrices. Highly water soluble drugs include salts formed with inorganic and organic acids (positively charged due to non-covalently attached protons), permanently positively (or negatively) charged molecules, and negatively charged molecules (salts of weak and strong acids). For example, highly water soluble drug means that its solubility is over 10 mg/mL, more preferably over 100 mg/mL.

Specific active agents suitable for use in formulations of this invention, micelle forming drug having a charge, can be selected based on critical micelle concentration (CMC) and/or log P, which are closely related (see, Example 3). Log P, the drug distribution coefficient between octanol and water, reflects the hydrophobic properties of the uncharged drug form. CMC, a measure of the concentration at which a particular compound will form a micelle, is a function of hydrophobicity, as well as molecular stereochemistry, group rotation ability, and counter ions. The presence of micelle-like charged drug aggregates within a hydrogel matrix containing oppositely charged polymers leads to cooperative interaction. It is this cooperative interaction that governs the release rate of drug from the polymeric matrix. Therefore, CMC and log P can be used to predict drug release rate and thus identify those drugs which will have extended release in formulations of this invention. Drugs with a low CMC and/or high log P would be released slowly in formulations of this invention, while those less likely to form micelles would be released with profiles similar to those for standard oral formulations.

Accordingly, the release profile of a drug can be modulated using any standard methods known to those of skill in the art to modulate the critical micelle concentration and/or the degree of cooperative interaction between a micelle-forming drug and the oppositely charged polymers. Methods of modulating CMC and/or the degree of cooperative interaction would include altering the hydrophobicity of the drug by addition of functional groups and any other techniques to alter electrostatic interaction between the drug and the polymeric excipient. In certain aspects, the present invention provides a method for extending the release profile of a micelle forming drug, comprising: decreasing the critical micelle concentration of the micelle forming drug, thereby extending the release profile of the micelle forming drug.

In certain other aspects, the present invention provides further methods of extending the release profile of a micelle forming drug. These include for example, varying the polymer compositions, changing the polymer-drug ratio, varying the additional amount of polymer having opposite charge as well as varying the tablet size and shape.

One method to determine whether micelles exist, is to measure the variation of light scattering at an angle of 90° i.e., S90, as a function of concentration in an appropriate solution. Thereafter, scattering graphs can be analyzed. If scattering is increasing continuously with increasing the concentration, no micelle formation is occurring. If graphs indicate clearly defined inflection in the S90 vs. concentration plots, it is attributed to the micelle formation. Critical micelle concentration is determined from the inflection point of graphs of the scattering at an angle of 90° to the incident beam, S90, as a function of the molar concentration. Those of skill in the art will know of other methods to determine micelle formation.

Advantageously, drug loads for formulations of this invention can be extremely high. Moreover, the release rate does not increase significantly with increase of drug content (e.g., up to 60 wt. %) in SGF and actually decreases with increase of drug content in SIF (see, Example 8).

In certain preferred aspects, the micelle forming drug has a positive charge or a negative charge at physiological pH. As used herein, physiological pH is about 0.5 to about 8, more preferably, about 0.5 to about 5.5. The positive charge or negative charge at physiological pH refers to the overall charge on the molecule. That is, it is possible to have more than one functional group contributing to the charge, as long as the overall charge is positive or negative.

One assay method to determine whether the micelle forming drug or polymer has a positive charge or a negative charge at physiological pH is to empirically determine the charge on the molecule. For example, a suitable buffer solution or gel is made having a certain pH. A cathode and an anode are placed in the buffered solution or, alternatively, a gel electrophoresis is used. The micelle forming drug if positively charged migrates to the cathode. If the micelle forming drug is negatively charged, the drug migrates to the anode. The polymer having an opposite charge in the pharmaceutical formulation will migrate to the opposite electrode. For example, if the micelle forming drug is positively charged, it will migrate to the cathode. The polymer having an opposite charge will migrate to the anode.

In another assay method, the charge on the micelle forming drug and/or polymer is assessed using the Henderson-Hasselbach equation. The Henderson-Hasselbach equation is a mathematical statement which defines the pH of a solution of a conjugate acid-base pair in terms of the dissociation constant of the weak acid and the equilibrium concentrations of the acid and its conjugate base. When pK=pH, then, [Ha] is equal to [A]. Values of pK yield quantitative information concerning acid strength, very strong acids being characterized by undefined pK values (pK=−log 0, example HCl); semi-strong acids being characterized by small pK values; and weak acids being characterized with large pK values. Using the Henderson-Hasselbach equation, the charge on the micelle forming drug and/or polymer is assessed to determine the charge thereon.

II. Charged Polymeric Excipients of the Invention

The formulation of this invention also comprises at least one polymeric excipient or polymer with a charge opposite that of the micelle-forming drug of the invention. In a preferred aspect, the cooperative interaction of the charged excipient with the micelle-forming drug is the basis for the extended release properties of this invention.

The formulation can comprise negatively charged polymers, such as ones with a carboxylic group or a sulfate group. These include, but are not limited to, sulfated polymers, polyacrylic acid, polymethacrylic acid, methylmethacrylic-methacrylic acid copolymer, alginates, xanthan gum, gellan gum, guar gum, carboxymethylcellulose, locust bean gum, and hyaluronic acid.

Especially preferred polymers with a negative charge include polyacrylic acid and sulfated polymers. Sulfated polymers include carrageenan (e.g., Viscarin® and/or Gelcarin®), and dextran sulfate. Preferably, when polyacrylic acid is selected as one polymer, sulfated polymers can be selected as other polymers.

Preferably, the formulation can also comprise a hydrogel-forming polymer with physical characteristics, such as high viscosity upon gelation, which permit the preparation of the present invention to withstand the contractile forces of the digestive tract associated with the digestion of food and more or less retain its shape during its travel down to the lower digestive tract, namely the colon. For example, a polymer showing a viscosity of not less than 1000 cps in 1% aqueous solution (at 25° C.) is particularly preferred.

The properties of the polymer depend on its molecular weight. The hydrogel-forming polymer which can be used in the present invention is preferably a substance of comparatively high molecular weight, viz. a polymer having an average molecular weight of not less than 2×10⁶ and more preferably not less than 4×10⁶. Further, the polymers can be branched chain, straight chain, crossed linked or any combination thereof.

Examples of said polymer substance are polyethylene oxide, such as POLYOX® WSR 303 (viscosity-average molecular weight: 7,000,000, viscosity: 7,500 to 10,000 cps (aqueous 1% solution at 25° C.)), POLYOX® WSR Coagulant (viscosity-average molecular weight: 5,000,000, viscosity: 5,500 to 7,500 cps (aqueous 1% solution at 25° C.)), POLYOX® WSR-301 (viscosity-average molecular weight of 4,000,000, viscosity: 1650-5500 cps (aqueous 1% solution at 25° C.)), POLYOX® WSR N-60K (viscosity-average molecular weight: 2,000,000, viscosity: 2,000 to 4,000 cps (2% aqueous solution at 25° C.) (all made by Union Carbide), ALKOX® E-75 (viscosity-average molecular weight: 2,000,000 to 2,500,000, viscosity: 40 to 70 cps (aqueous 0.5% solution at 25° C.)), ALKOX® E-100 (viscosity-average molecular weight of 2,500,000 to 3,000,000, viscosity: 90 to 110 cps (aqueous 0.5% solution at 25° C.)), ALKOX® E-130 (viscosity-average molecular weight: 3,000,000 to 3,500,000, viscosity: 130 to 140 cps (aqueous 0.5% solution at 25° C.)), ALKOX® E-160 (viscosity-average molecular weight: 3,600,000 to 4,000,000, viscosity: 150 to 160 cps (aqueous 0.5% solution at 25° C.)), ALKOX® E-240 (viscosity-average molecular weight: 4,000,000 to 5,000,000, viscosity: 200 to 240 cps (aqueous 0.5% solution at 25° C.)) (all made by Meisei Kagaku Co., Ltd.), PEO-8 (viscosity-average molecular weight: 1,700,000 to 2,200,000, viscosity: 20 to 70 cps (aqueous 0.5% solution at 25° C.)), PEO-15 (viscosity-average molecular weight: 3,300,000 to 3,800,000, viscosity: 130 to 250 cps (aqueous 0.5% solution at 25° C.)), PEO-18 (viscosity-average molecular weight: 4,300,000 to 4,800,000, viscosity: 250 to 480 cps (aqueous 0.5% solution at 25° C.)) (all made by Seitetsu Kagaku Kogyo Co., Ltd.), etc.

In order to provide a hydrogel-type preparation suitable for sustained release, it is generally preferable that the preparation contains about 10 to about 95 weight %, more preferably, about 15 to about 90 weight % of a hydrogel-forming polymer of a preparation weighing less than 600 mg. Preferably, the preparation contains not less than 70 mg per preparation and preferably not less than 100 mg per preparation of the hydrogel-forming polymer. The above-mentioned levels will insure that the formulation will tolerate erosion in the digestive tract for a sufficiently long time in order to achieve sufficient sustained release.

The above hydrogel-forming polymer may be used singly, or two or more kind(s) of the above hydrogel-forming polymers in mixture may be used.

Preferably, the particular combination and ratio of polymeric excipients is that which allows the slowest rate of release under both gastric and intestinal conditions, pH independently. The optimal combination and ratio can vary depending on the particular active agent and percent loading of active agent.

Preferred combinations of excipients includePAA/PEO, PAA/carrageenan, and PAA/dextran sulfate. Preferably, the polymers are in a 1:0.5 ratio, 1:1 ratio, or a 1:5 ratio; most preferably, the polymers are in a 1:1.5 ratio.

Preferred combinations of excipients also include PAA/carrageenan/PEO. Preferably, PAA and carrageenan are in a 1:0.5 ratio, 1:1 ratio, or a 1:5 ratio; most preferably, the polymers are in a 1:1.5 ratio. Preferably, PAA plus carrageenan, and PEO are in a 1:0.5 ratio, 1:1 ratio, or a 1:2 ratio; most preferably, the polymers are in a 1:1.5 ratio.

In order for accomplishment of sustained drug release in the lower digestive tract as well as in upper digestive tract of humans, the preparation should be a gelled at least 2 hours after administration and the tablet should be further eroded through moving the lower digestive tract so that the tablet is released.

The term “percentage gelation of the formulation” used in the present invention means the ratio of the tablet that has been gelled once the compressed tablet has been moistened for a specific amount of time and is determined by the method of determination of the percentage gelation described below (see, Test Method 2). Because the preparation absorbs water when retained in the upper digestive tract and thereby almost completely gels (that is, percentage gelation is not less than 70%, preferably not less than 75%, more preferably not less than 80%) and move to the lower digestive tract as the surface of the preparation is being eroded with drug being released by further erosion, the drug is continually and thoroughly released and absorbed. As a result, sustained release performance is realized, even in the lower digestive tract where there is little water. Specifically, if the percentage gelation is less than approximately 70%, sufficient release of the drug will not be obtained and there is a chance of a reduction in bioavailability of the drug (EP No. 1,205,190A1).

The term “upper digestive tract” in the present invention means the part from the stomach to the duodenum and jejunum “lower digestive tract” means the part from the ileum to the colon.

The formulation can also comprise hydrophilic base to achieve the higher percent gelation. There are no particular restrictions to the hydrophilic base as long as it can be dissolved before above-mentioned hydrogel-forming polymer substance gels. For example, the amount of water needed to dissolve 1 g of this hydrophilic base is preferably 5 mL or less (at 20±5° C.), more preferably 4 mL of less (at same temperature).

Examples of said hydrophilic base include water-soluble polymers such as polyethylene glycol (for instance, Macrogol 4000, Macrogol 6000 and Macrogol 20000, all of which are trade names of Nippon Oil and Fats Co.), polyvinyl pyrrolidone (for instance, PVP® K30, of which is trade name of BASF), sugar alcohols, such as D-sorbitol, xylitol, etc., saccharides, such as sucrose, maltose, lactulose, D-fructose, dextran (for instance, Dextran 40), glucose, etc., surfactants, such as polyoxyethylene hydrogenated castor oil (for instance, Cremophor® RH40 (made by BASF), HCO-40, HCO-60 (made by Nikko Chemicals), polyoxyethylene polyoxypropylene glycol (for instance, Pluronic® F68 of which is trade name of Asahi Denka), etc. Polyethylene glycol, sucrose, and lactulose are preferred and polyethylene glycol (particularly Macrogol 6000) is further preferred. The above hydrophilic base can be used singly, or two or more kind(s) of the above hydrophilic base in mixture can be used.

When the hydrophilic base is added in the present invention, the ratio used is preferably approximately 5 to approximately 80 wt % per total preparation, more preferably 5 to 60 wt % based on the total preparation.

Preferred combinations of excipients include PAA/PEO/PEG. Preferably, PAA and PEO are in a 1:0.5 ratio, 1:1 ratio, or a 1:5 ratio. More preferably, the amount of PEG is 5 wt. % to 60 wt. % based on the total preparation

Preferred combinations of excipients also include PAA/carrageenan/PEO/PEG. Preferably, PAA and carrageenan are in a 1:0.5 ratio, 1:1 ratio, or a 1:5 ratio. Preferably, PAA plus carrageenan, and PEO are in a 1:0.5 ratio, 1:1 ratio, or a 1:2 ratio. More preferably, the amount of PEG is 5 wt. % to 60 wt. % based on the total preparation.

The formulation can also comprise a single positively charged polymer or combinations of such polymers, including, but not limited to, polyethylene imine, chitosan, polyvinylpirridinium bromide, and polydimethylaminoethylmethacrylate.

Depending on the polymer(s) viscosity, the polymer material can form a matrix comprising the active ingredient. For example, a polymer showing a viscosity of not less than 1000 cps in 1% aqueous solution is particularly preferred due to its matrix forming ability.

Extending release of a micelle forming drug can be achieved by a method of oral administrating formulation of this invention.

III. Other Tablet Modifications

Modification of drug release through the tablet matrix of the present invention can also be achieved by any known technique, such as, e.g., application of various coatings, e.g., ion exchange complexes with, e.g., Amberlite IRP-69. The tablets of the invention can also include or be co-administered with GI motility-reducing drugs. The active agent can also be modified to generate a prodrug by chemical modification of a biologically active compound which will liberate the active compound in vivo by enzymatic or hydrolytic cleavage, etc. Additional layers or coating can act as diffusional barriers to provide additional means to control rate and timing of drug release.

IV. Formulation Additives

If desired, the preparation of the present invention may include appropriate amounts of other pharmaceutically acceptable additives such as vehicles (e.g., lactose, mannitol, potato starch, wheat starch, rice starch, corn starch, and crystalline cellulose), binders (e.g., hydroxypropylmethylcellulose, hydroxypropylcellulose, methylcellulose, and gum arabic), swelling agents (e.g., carboxymethylcellulose and carboxy-methylcellulose calcium), lubricants (e.g., stearic acid, calcium stearate, magnesium stearate, talc, magnesium meta-silicate aluminate, calcium hydrogen phosphate, and anhydrous calcium hydrogen phosphate), fluidizers (e.g., hydrous silica, light anhydrous silicic acid, and dried aluminum hydroxide gel), colorants (e.g., yellow iron sesquioxide and iron sesquioxide), surfactants (e.g., sodium lauryl sulfate, sucrose fatty acid ester), coating agents (e.g., zein, hydroxypropylmethyl-cellulose, and hydroxypropylcellulose), buffering agents (e.g., sodium chloride, magnesium chloride, citric acid, tartaric acid, bibasic sodium phosphate, monobasic sodium phosphate, calcium hydrogen phosphate, ascorbic acid, ), aromas (e.g., l-menthol, peppermint oil, and fennel oil), preservatives (e.g., sodium sorbate, potassium sorbate, methyl p-benzoate, and ethyl-benzoate).

V. Manufacturing

The preparation of the present invention is a solid preparation having a certain shape, and can be manufactured by any conventional processes. Typical processes include, e.g., compression tableting manufacturing processes. These processes comprise blending and if necessary granulating the active agent, the charged polymers, and if desired, additional additives, and compression-molding the resulting composition/formulation. Alternative processes include, e.g., a capsule compression filling process, an extrusion molding process comprising fusing a mixture and setting the fused mixture, an injection molding process, and the like. Any coating treatments, such as, e.g., sugar coating, may also be carried out.

The following examples are intended to illustrate, but not to limit, the present invention.

### EXAMPLES

### Test Method 1

This Test Method illustrates the basic procedure for manufacturing formulations of this invention, as well as measuring drug release.

Several different formulations with different drugs were manufactured. Drugs were manually mixed with the excipients in a mortar and compressed into 400 mg tablets using Carver press or Oil press with 1000 lb applied force. Flat face 11 mm round tooling was used.

Materials

Carbopol 971 (BF Goodrich); Polyox 303 (Union Carbide); two types of carrageenan, Viscarin® 109 and Gelcarin (FMC); Xantural™ 180 (Monsanto Pharmaceutical Ingredients), a xanthan gum Keltone® LVCR (Monsanto Pharmaceutical Ingredients); a sodium alginate Chitosan (M. W. International , Inc.); Macrogol 6000 (Nippon Oil and Fats Co.); Methocel K100M (The Dow Chemical Company); a hydroxypropylmethylcellulose (HPMC); Cellulose Gum 12M31P TP (Hercules); a sodium carboxymethylcellulose (CMC); and Dextran Sulfate (Sigma).

Methods

In vitro drug release was measured by in vitro dissolution experiments. These studies were carried out using USP apparatus II at a paddle speed of 100 rpm in 1000 ml dissolution medium from Examples 1 to 10. Drug release was evaluated with either Simulated Gastric Fluid (SGF), pH=1.2 or Simulated Intestinal Fluid (SIF), pH=7.5, both prepared according to USP without enzyme added. Tablet sinkers were applied in all experiments. At predetermined time intervals, a sample was withdrawn from the vessel and assayed using a UV-VIS spectrophotometer at a wavelength of 240 nm.

### Example 1

This example illustrates that drug release rate does not correlate with drug solubility, indicating that a specific interaction is influencing its release rate.

The release behavior of a large group of basic highly soluble drugs (10 wt. % of drug ) from a directly compressed matrix tablet using 1:1.5 polyacrylic acid/polyethylene oxide (PAA/PEO) mix as excipient was studied under modified Simulated Intestinal Fluid (SIF) conditions. Release rate was characterized by T₅₀ (time during which 50% of drug has been released from matrix to the solution) (FIG. 1). Results of the study are presented in Table 1, where drug properties and release rate are summarized.

Identically charged drugs have significantly different release profiles in modified SIF which do not correlate with drug solubility (FIG. 1, Table1). Therefore, it can be concluded that a single electrostatic interaction does not by itself result in extended release of soluble drugs.

### Example 2

This example illustrates that the log P of a drug can be used to predict whether extended release will be achieved using the formulation of this invention. An ability to predict drug release behavior based on the log P characteristic is one of the key advantages of this invention.

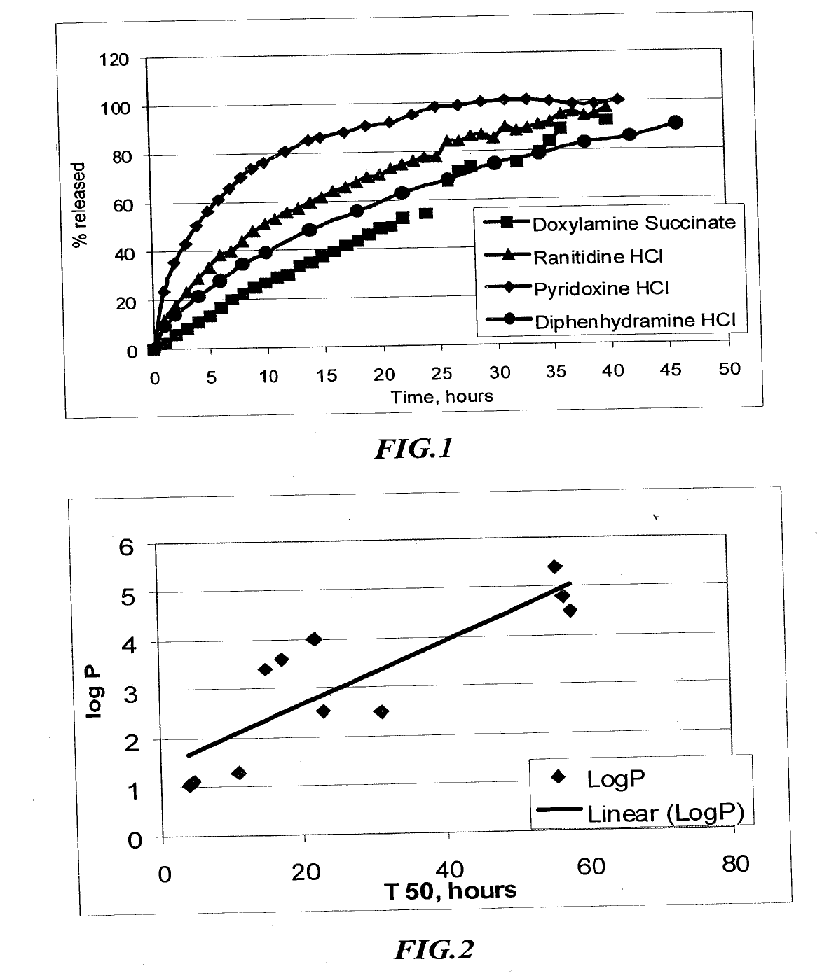

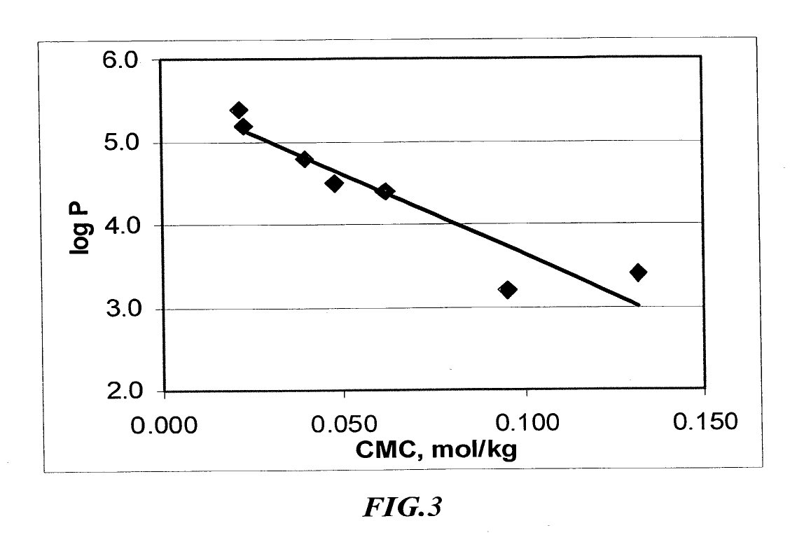

The ability of a drug to bind to a particular polyelectrolyte is dependent on its critical micelle concentration (CMC). However, since the CMC value is rarely available for drugs, an attempt was made to relate the release rate to drug properties which are commonly used for drug characterization. For drugs which were used in the above-described release experiments (Table 1), a variety of parameters such as molecular weight, solubility, pKa, log P, log D, and surface tension were analyzed in terms of their correlation with the release time. It appeared that log P (distribution coefficient of uncharged drug form between octanol and water) demonstrates a close to linear relationship with T₅₀ (FIG. 2). Log P is closely related to CMC. In fact, a practically linear relationship has been established between log P and CMC (FIG. 3). Log P and CMC values for different drugs were extracted from the Attwood publications.

### Example 3

This example illustrates that extended release can be achieved for permanently positively charged molecules using a 1:1.5 PAA/PEO excipient mixture.

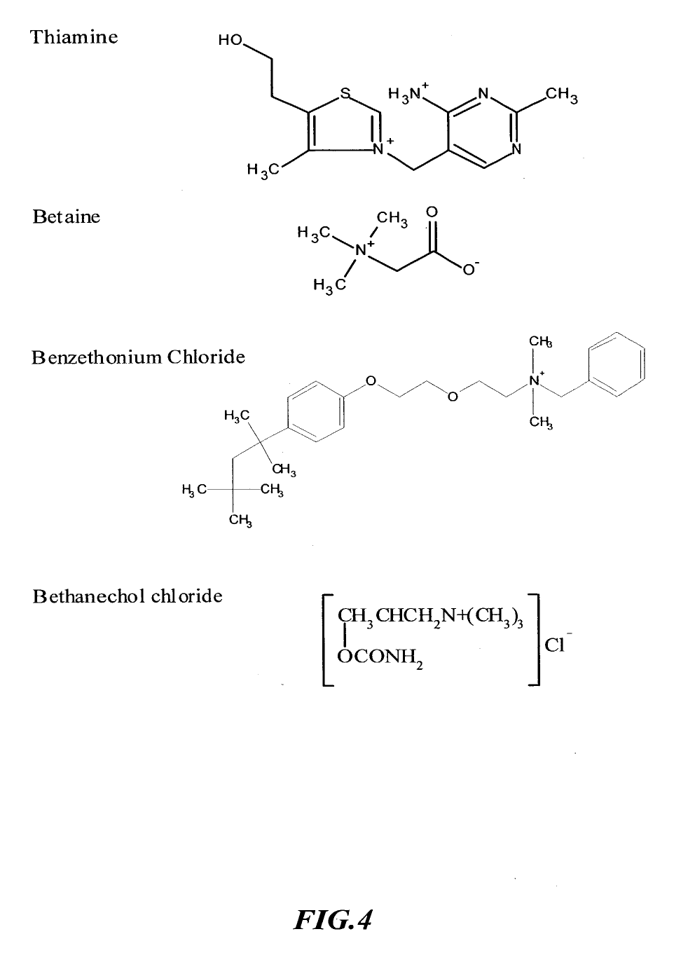

The following positively charged molecules were tested: benzethonium chloride and bethanechol chloride, which have one positive charge; thiamine mononitrate and thiamine hydrochloride, which have two positive charges; and betaine, which is a dipole (FIG. 4). Although thiamine HCl showed slightly fast release, all the drugs demonstrated extended release with different rates (Table 2).

TABLE 1
Model drug characteristics

| | | Solubility, | | |
|---|---|---|---|---|
| Name | MW | mg/ml | T₅₀ | Log P |
| Pyridoxine HCl | 205.64 | 222 | 4 | −1.9 |
| Pseudoephedrine HCl | 201.73 | ˜250 | 4 | 1.0 |
| Cevimeline HCl | 244.79 | 766 | 4.5 | 1.1 |
| Ranitidine HCl | 350.91 | ˜200 | 11 | 1.3 |
| Diphenhydramine HCl | 291.9 | 1000 | 15 | 3.4 |
| Diltiazem HCl | 450.98 | 800 | 18 | 3.6 |
| Doxylamine Succinate | 388.8 | 1000 | 23 | 2.5 |
| Tramadol HCl | 299.8 | >1000 | 31 | 2.5 |
| Amitriptuline HCl | 313.9 | 500 | 57 | 4.8 |
| Chlorpromazine HCl | 354.4 | 400 | 56 | 5.4 |
| Imipramine HCl | 332.9 | 500 | 58 | 4.5 |
| Benoxinate HCl | 344.9 | 1000 | 22 | 4.0 |

These results demonstrate that even if a drug does not have strong hydrophobic groups, specific interaction with charged polymeric excipients is still possible (see, for example bethanechol chloride), as long as the drug carries a permanent positive charge. On the other hand, drug structure and charge location can play an important role in the ability to interact with polymeric excipients (see, thiamine HCl). Thiamine's location of charges at the center of the molecule (FIG. 4) may effect micelle formation.

### Example 4

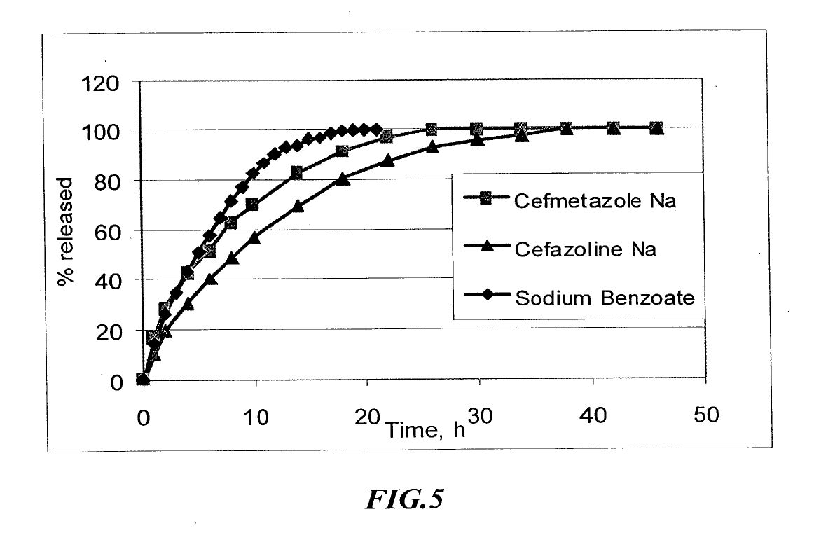

This example illustrates that oppositely charged drugs and polymeric excipients are critical for extending drug release. As FIG. 5 shows, the highly soluble negatively charged drugs, sodium cefazoline and sodium cefmatazole, diffuse out the negatively charged PAA/PEO matrix with a T₅₀ of about 5 hours without achieving extended release.

### Example 5

This example demonstrates the effect of fluid environment on drug release profiles for 1:1.5 PAA/PEO mixtures.

The initial experiments described in Examples 1-4 were conducted under Simulated Intestinal Fluid (SIF) conditions, where PAA is ionized. To evaluate the release kinetics under gastric conditions, dissolution of different types of soluble drugs was performed in modified Simulated Gastric Fluid (SGF). Table 3 compares T₅₀ values in SGF and SIF for different drugs.

TABLE 2
Positively charged drug characteristics and T₅₀.

| | Solubility, | T50, | T10, | |
|---|---|---|---|---|
| Name | mg/ml | h | h | Comments |
| Thiamine Mononitrate | 300 | 14 | 1 | Two positive charges |
| Thiamine HCl | 1000 | 7.5 | 1 | Two positive charges |
| Betaine | 650 | 22 | 6 | Dipole |
| Bethanechol Chloride | 1700 | 55 | 10 | Forms insoluble |
| | | | | complex with PAA |
| Benzethonium | 1000 | | 25 | Forms insoluble |
| Chloride | | | | complex with PAA |

TABLE 3
T₅₀ values in SGF and SIF.

| | T50 in SGF, | T50 in SIF, |
|---|---|---|
| Drug Name | hours | hours |
| Diltiazem HCl | 8 | 18 |
| Tramadol HCl | 8 | 31 |
| Diphenhydramine Citrate | 12 | 25 |
| Bethanechol Chloride | 24 | 55 |
| Betaine Hydrochloride | 4 | 22 |
| Thiamine Mononitrate | 3 | 14 |

As Table 3 shows, release time in SIF is significantly longer that in SGF. Obviously, in low pH media ionization of PAA is suppressed to a great extent. This may prevent formation of cooperative bonds between PAA/PEO and the drug. Another possible reason for the short release times in SGF is that formation of a hydrogen-bonded polymer complex between the electronegative oxygen atom of PEO and the carboxylic group of PAA at low pH conditions blocks the carboxylic groups from interaction with drug.

### Example 6

This example illustrates polymeric excipient combinations which provide sustained release under both SGF and SIF conditions.

Evaluation of Multiple Polysaccharides

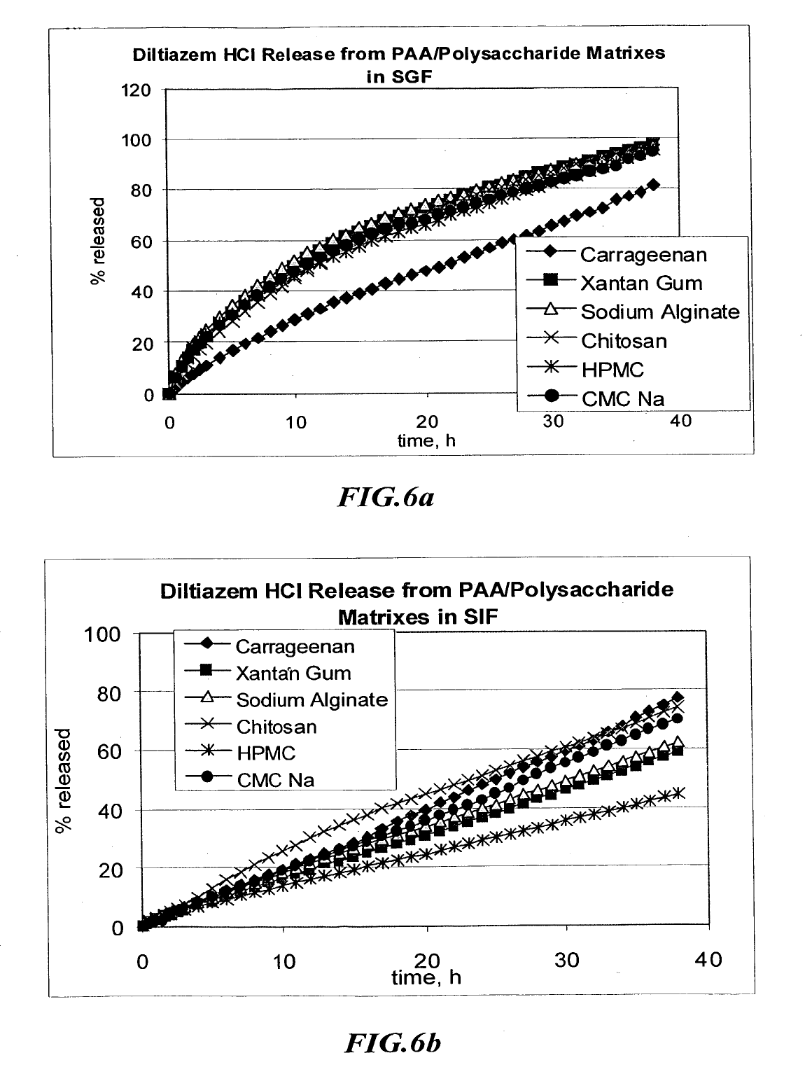

Drug release rates were tested for seven different polysaccharides (carrageenan, xanthan gum, sodium alginate, chitosan, HPMC, CMC-Na) combined with PAA, containing 25 wt. % Diltiazem HCl (DI) (FIG. 6). The results demonstrate that a combination of PAA with carrageenan can provide the slowest release of drug in SGF. This effect is probably due to the strong acidic nature of carrageenan functional groups (—SO₄⁻) which stay negatively charged even at low pH conditions and enable interaction between the carrageenan and a drug.

Evaluation of other Sulfated Polymers

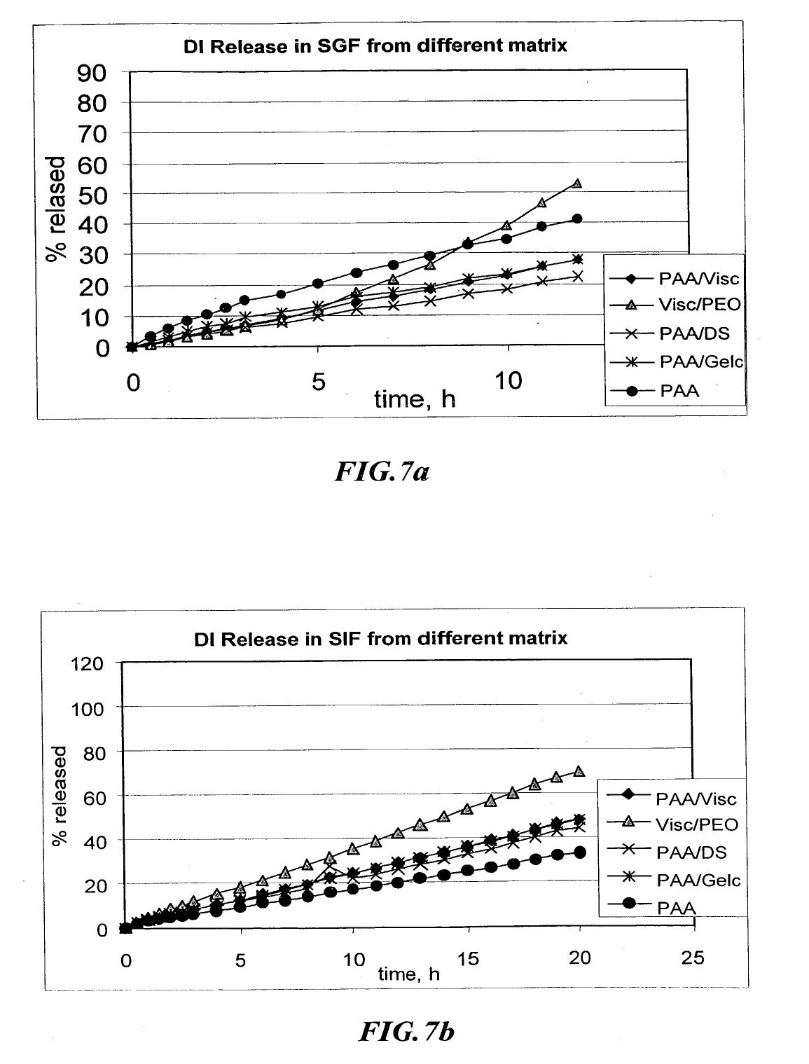

Different types of carrageenan, as well as dextran sulfate, were used in combination with PAA or PEO at a 1:1 ratio and Diltiazem HCl (25 wt. %) and release rate was measured in SGF and SIF. Extended release was observed for all combinations containing sulfated polymers (FIG. 7).

Further Analysis of Effect of Substitution of PAA for PEO

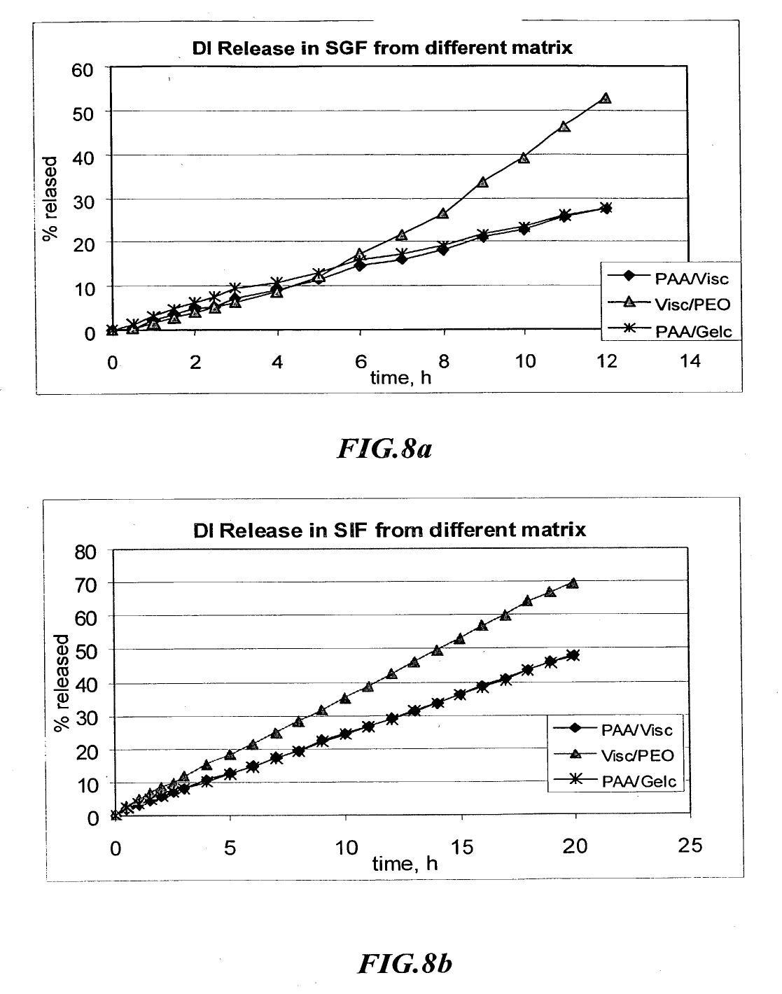

When PAA in PAA/Carrageenan (1:1) formulation is substituted by high MW PEO, release profiles for PAA/Carrageenan (1:1) and PEO/Carrageenan (1:1) formulations in SGF overlap for about 6 hours (FIG. 8a). After this time, fast matrix erosion causes faster drug release from PEO/Carrageenan matrix. In contrast, in SIF, PAA/Carrageenan formulation demonstrates slower drug release than PEO/Carrageenan formulation over the entire period of time (FIG. 8b). Therefore, combination of PAA and Carrageenan can provide the best in vitro drug release characteristics in both SGF and in SIF.

Comparison of PAA/Carrageenan Release Rates in SIF and SGF

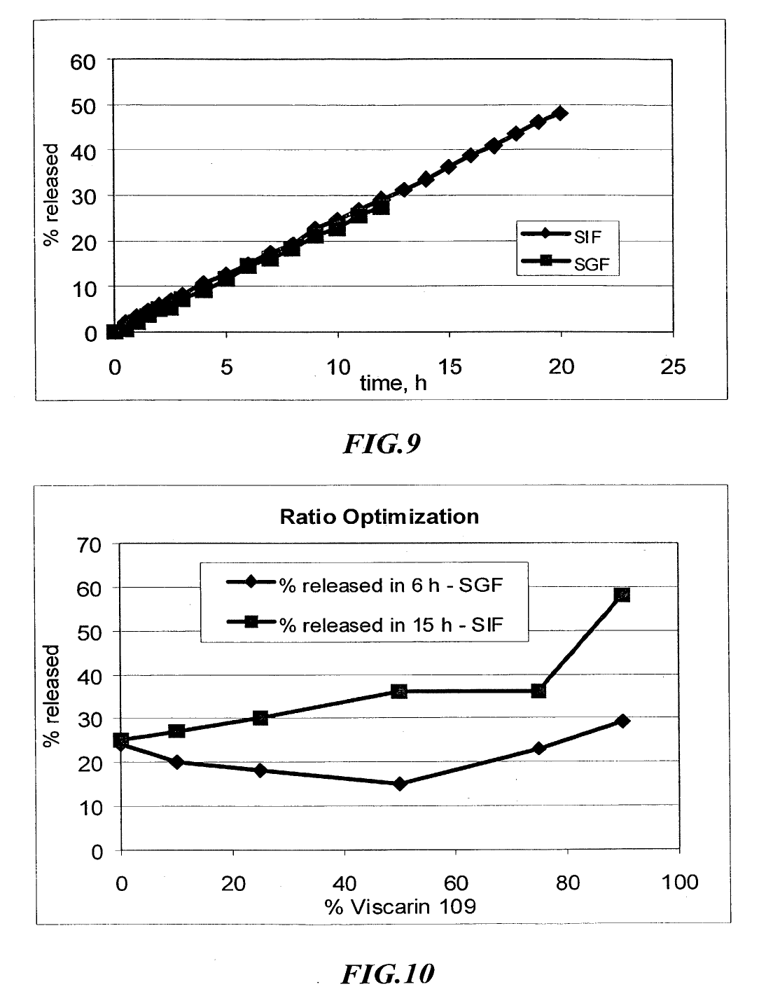

FIG. 9 demonstrates that DI (25 wt. %) release from the PAA/Carrageenan (1:1) matrix is linear in both SGF and SIF and that release rates in the two media are identical. A dissolution test for samples where the media was switched after 2 hours resulted in a linear release profile very close to the profiles in FIG. 9.

### Example 7

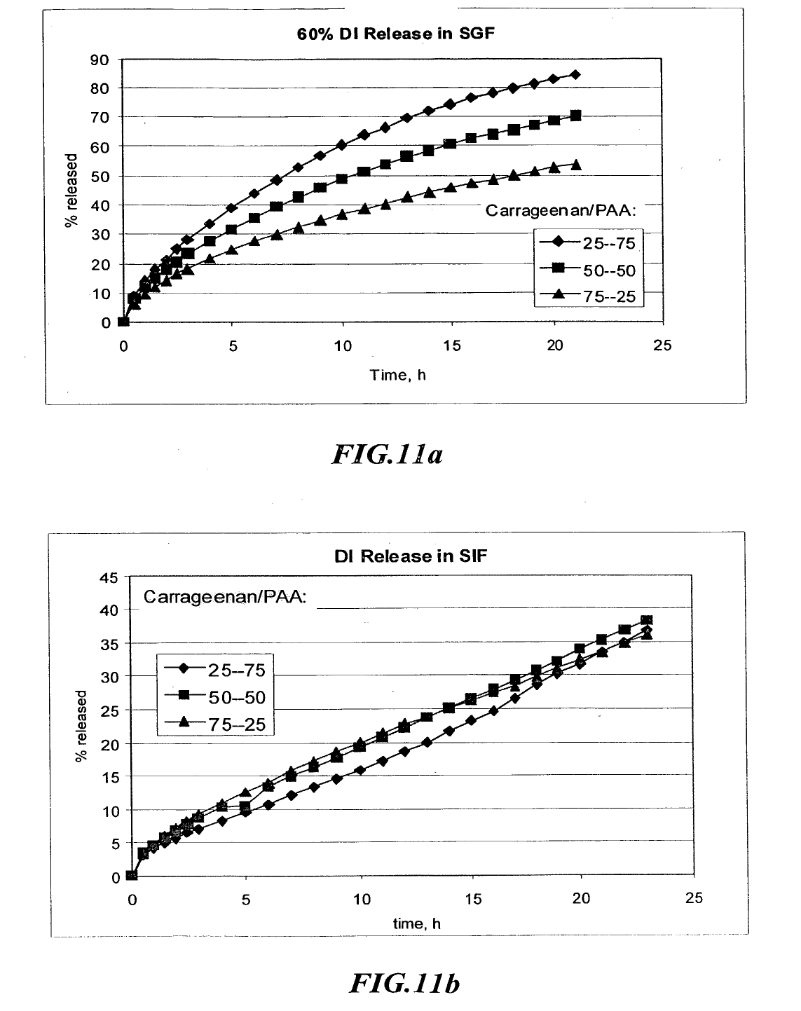

This example illustrates that the optimal polymer excipient composition is media dependent (FIG. 10).

For the formulation containing 25 wt. % DI, the lowest release rate in SGF was achieved with 1:1 PAA/Carrageenan composition. In SIF, release rate decreased with increasing amounts of PAA in the formulation.

Interestingly, different optimal compositions were observed for a formulation with high DI content (60 wt. %). In SGF, the drug release rate decreased with an increase in carrageenan content and in SIF, the release rate was practically independent from excipient ratio (FIG. 11).

Based on these observations, we believe that the release behavior is most likely governed by drug/excipient complex stoichiometry in different media.

### Example 8

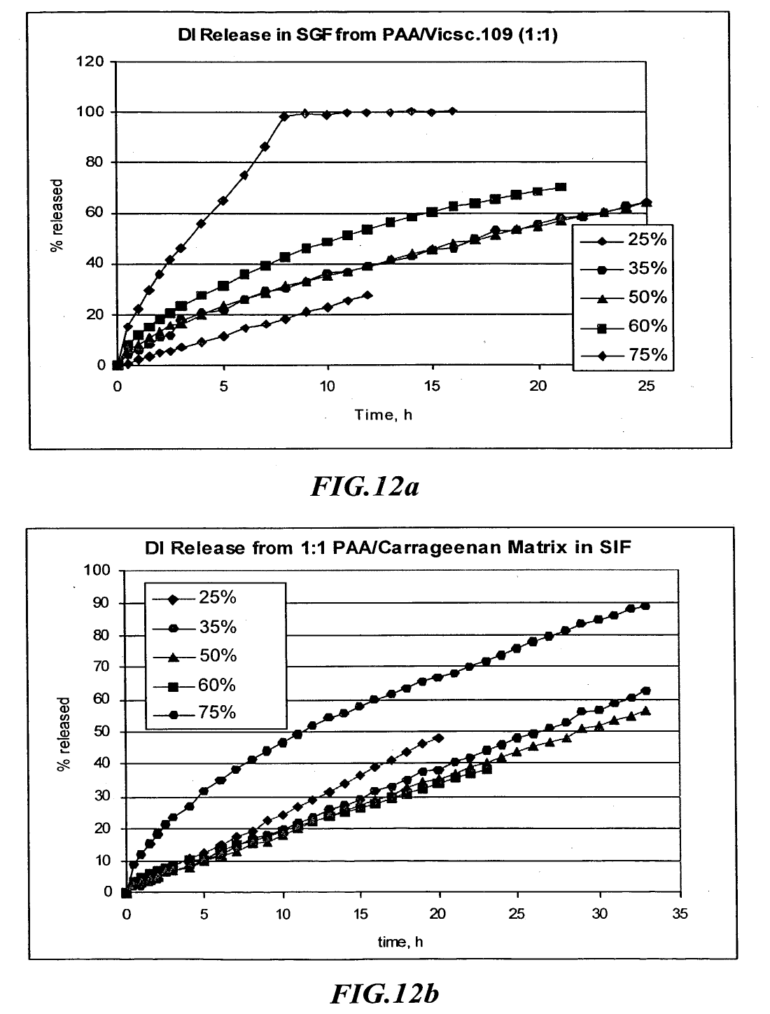

This example illustrates that an increase in drug loading has an insignificant effect on the release rate in SIF for drug loading up to 60 wt %. In SGF, the increase in release rate is relatively small for drug loads up to 50 wt. % (FIG. 12).

### Example 9

This example illustrates the superior ability of the formulations of this invention to extend drug release.

DI (25%) release from PAA/Carrageenan (1:1) matrix was compared with previously described formulations containing PAA and Carrageenan (Bonferoni et al., AAPS Pharm. Sci. Tech, 1(2) article 15 (2000); Bubnis et al., Proceed. Int'l. Symp. Control. Rel. Bioact. Mater., 25, p. 820 (1998); Devi et al., Pharm. Res., v.6, No 4, 313-317 (1989); Randa Rao et al., J. Contr. Rel., 12, 133-141 (1990); Baveja et al., Int. J. Pharm., 39, 39-45 (1987); Stockwel et al., J. Contr. Rel. 3, 167-175 (1986); Perez-Marcos et al., J. Pharm. Sci., v.85, No. 3 (1996); Perez-Marcos et al., Int. J. Pharm. 111, 251-259 (1994); Dabbagh et al., Pharm. Dev. Tech., 4(3), 313-324 (1999); Bonferoni et al., J. Contr. Rel. 25, 119-127 (1993); Bonferoni et al., J. Contr. Rel. 30, 175-182 (1994); Bonferoni et al., J. Contr. Rel. 51, 231-239 (1998); U.S. Pat. No. 4,777,033; EU Patent 0 205 336 B1).

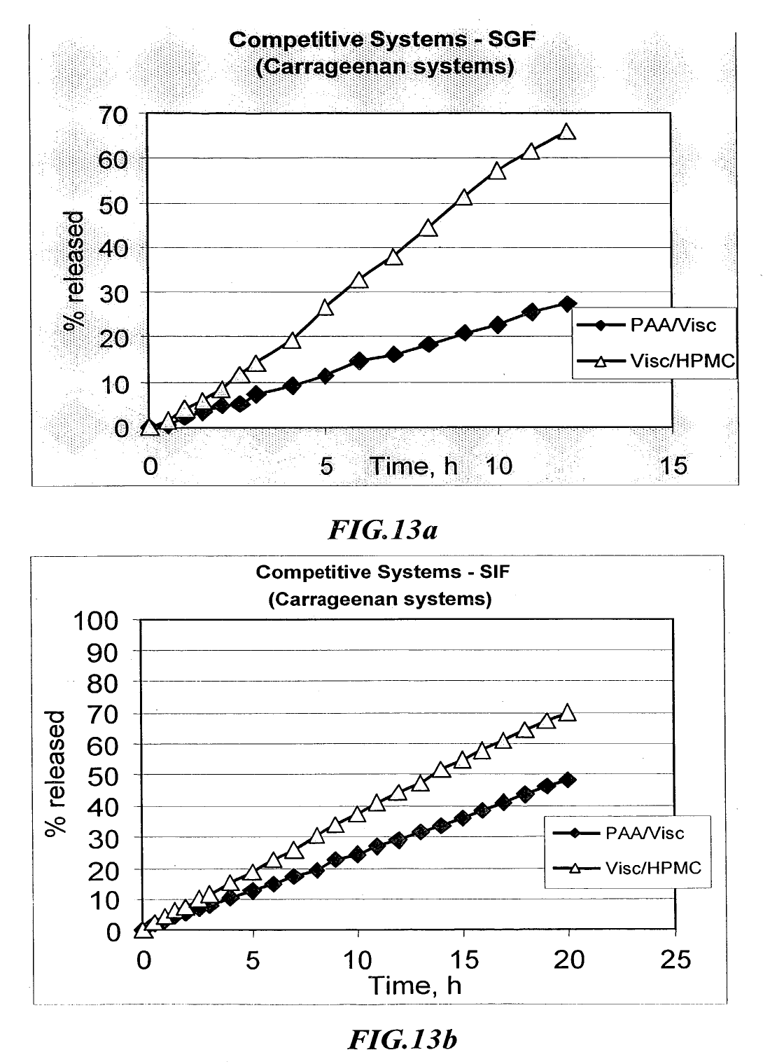

Carrageenan-containing systems described in the literature include carrageenan/HPMC and carrageenan/CMC. All matrices were prepared in the same way as the Viscarin 109/second polymer (1:1) mix. Formulations with the PAA/carrageenan (1:1) matrix demonstrated significantly slower DI release both in SGF and in SIF (FIG. 13).

An extended release system with PAA/HPMC (U.S. Pat. No. 4,777,033; EU Patent 0 205 336 B1) has been described.

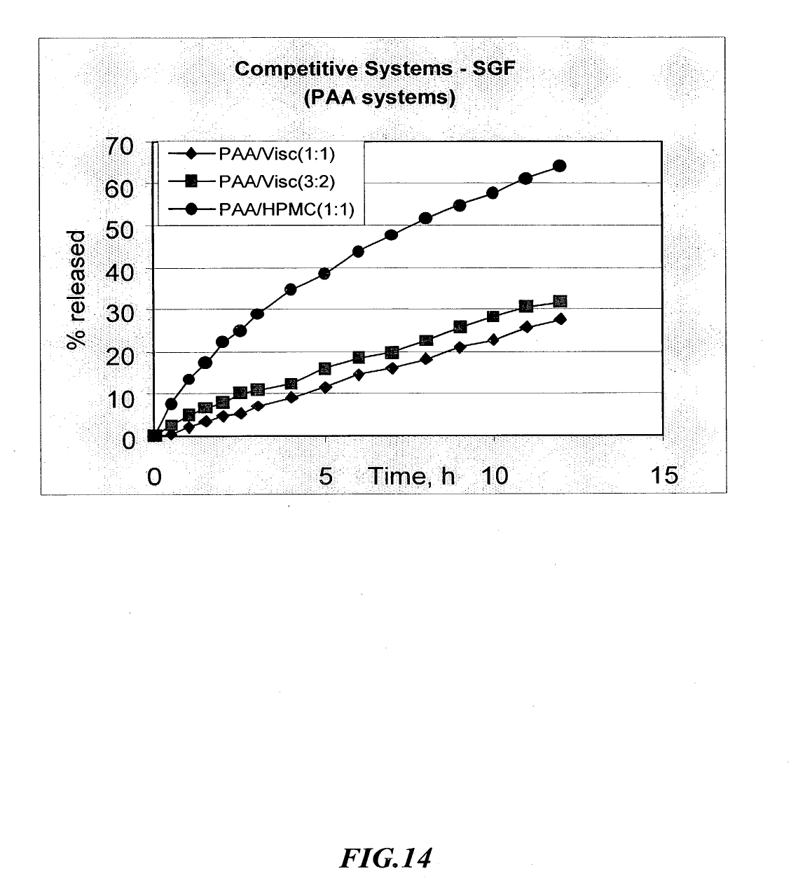

Formulations with the PAA/carrageenan (1:1 and 3:2) matrix demonstrated significantly slower DI release than that with PAA/HPMC (1:1) as a control in SGF (FIG. 14), although all preparations indicated an extended drug release in SIG with a T₅₀ of mo re than 20 h.

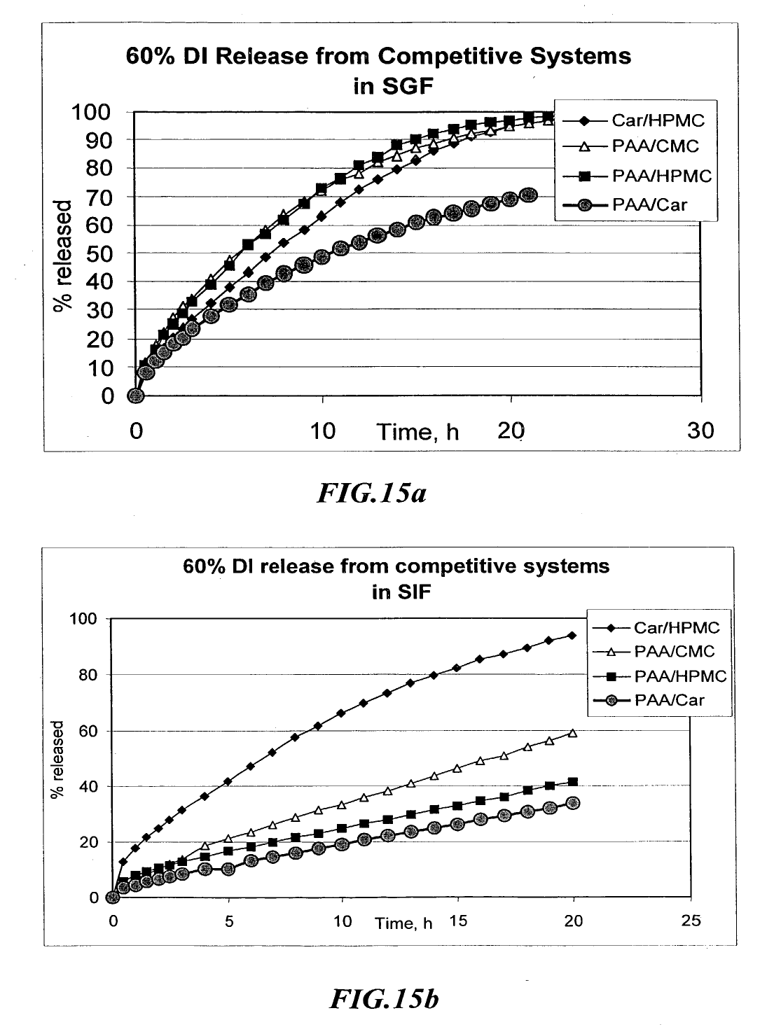

When the amount of drug in the system is increased to 60 wt. %, the release rate from PAA/Carrageenan system remains the slowest compared to all other competitive systems (FIG. 15).

### Example 10

This example compares release rates of various drugs for the original formulation (PAA/PEO) and the new PAA/carrageenan formulations.

Release rates of different drugs which previously demonstrated interaction with PAA/PEO matrix were compared to the release rates from the PAA/carrageenan (1:1) matrix. It appeared that most of the drugs show extended close to zero-order release from the PAA/carrageenan matrix. Typically, release of the drugs from PAA/carrageenan matrix was slower both in SGF and in SIF compare to the release from PAA/PEO matrix, although it was not the case for all the drugs.

To illustrate, the following Table 4 sets forth T₅₀ values (release times) in SIF. In this study, the PAA/PEO (1:1.5) formulation contained 10% of active and PAA/Carrageenan (1:1) formulation contained 25% of active.

TABLE 4
T50 values in SIF.

| Drug | PAA/PEO | PAA/Car | LogP |
|---|---|---|---|
| Thiamin HCl | 7.5 | 7.5 | |
| Ranitidine HCl | 11 | 15 | 1.3 |
| Diphenhydramine | 15 | 8 | 3.4 |
| HCl | | | |
| Diltiazem HCl | 18 | 21 | 3.6 |
| Benoxinate HCl | 22 | 23 | 5.2 |
| Naratriptan HCl | 22 | 25 | 1.8 |
| Doxylamine | 23 | 22 | 2.5 |
| Succinate | | | |
| Tamsulosin HCl | 26 | 25 | 2.24 |

### Test Method 2

Dissolution Test

In vitro drug release was measured by in vitro dissolution experiments. These studies were carried out using The Pharmacopeia of Japan XIV(referred to “JP” hereinafter) Dissolution Test Method 2 (paddle method) at a paddle speed of 200 rpm in 900 ml dissolution medium. Drug release was evaluated with either JP Disintegration Test Fluid 1 (referred to “JP 1st fluid” hereinafter), pH=1.2 or JP Disintegration Test Fluid 2 (referred to “JP 2nd fluid” hereinafter), pH=6.8. Tablet sinkers were not applied in the experiments. At predetermined time intervals, a sample was withdrawn from the vessel and assayed using a UV-VIS spectrophotometer at a wavelength of 250 m.

Gelation Test

Using JP 1st fluid and JP 2nd fluid, a gelation test was carried out as follows.

The test tablet was moistened for 2 hours in test medium at 37° C., gel layer was removed and the core portion not forming a gel was taken out, followed by drying at 40° C. for 5 days in a dryer and dried core was weighted (W_{obs}). The percent gelation of the formulations is calculated by means of Equation 1. The value obtained by subtracting core weight from initial tablet weight (Wᵢₙᵢₜᵢₐₗ) and dividing this by initial tablet weight is multiplied by 100 to calculate the percent gelation (G).

The “percent gelation” as used herein represents the percentage of the portion of the tablet which has undergone gelation. The method of calculating the percent gelation is not particularly limited but the following method may be mentioned as an example.

Thus, the test tablet is moistened for a predetermined time, the volume (or weight) of the portion not forming a gel is then measured and the result is subtracted from the volume (or weight) of the tablet before the beginning of the test.

Percent gelation (G, %)=(1−(W_{obs}−Wᵢₙᵢₜᵢₐₗ))×100 (Equation 1)

W_{obs}: The weight of the portion not gelled after initiation of the test

Wᵢₙᵢₜᵢₐₗ: The weight of the preparation before initiation of the test

### Example 11

This example illustrates the effect of additional amount of polymers having a charge opposite that of the micelle-forming drug on drug release profiles.

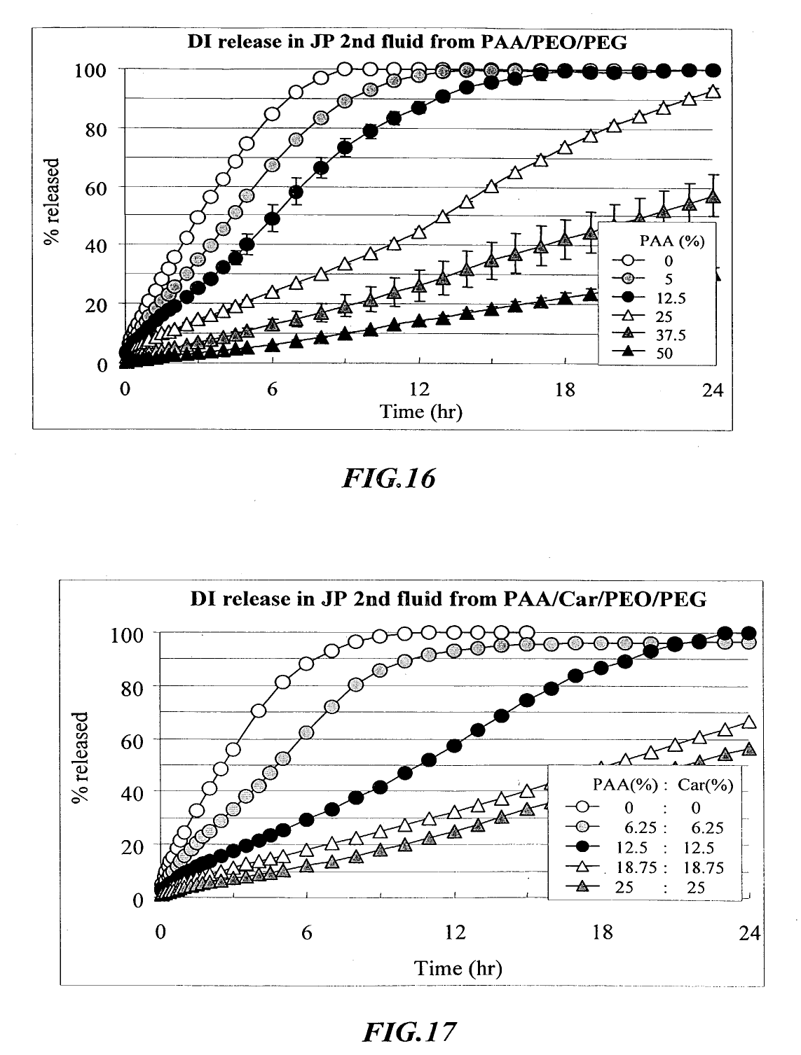

Different amount of PAA was used in combination with the mixture of PEO/PEG (1:1) at a 1:0 ratio (PAA wt. % to the total amount is 50), 1:1 ratio (PAA wt. % to the total amount is 25), 3:1 ratio (PAA wt. % to the total amount is 37.5), 1:3 ratio (PAA wt. % to the total amount is 12.5), or 1:9 ratio (PAA wt. % to the total amount is 5), containing 50 wt. % Diltiazem HCl. The Formulation comprising PEO/PEG at a 1:1 ratio without PAA, containing 50 wt. % Diltiazem HCl was prepared as a control. Drug release rate was evaluated in JP 2nd fluid according to the method as described in Test Method 2 (FIG. 16). Extended drug release was achieved for all preparations containing PAA, even in case of containing a small amount of PAA such as 5 wt. % of total preparation. The results also demonstrated the drug release rate decreased with increasing the amount of PAA instead of mixture of PEO/PEG (1:1).

The effect of additional amount of PAA and carrageenan mixture on drug release profiles was also investigated. The ratio of PAA and carrageenan, and the ratio of PEO/PEG was fixed 1:1, respectively. Different amount of PAA/carrageenan (1:1) was used in combination with the mixture of PEO/PEG (1:1) at a 1:0 ratio (both PAA and carrageenan wt. % to the total amount is 25 and 25, respectively), 3:1 ratio (both PAA and carrageenan wt. % to the total amount is 18.75 and 18.75, respectively), 1:1 (both PAA and carrageenan wt. % to the total amount is 12.5 and 12.5, respectively) ratio to 1:3 ratio(both PAA and carrageenan wt. % to the total amount is 6.25 and 6.25, respectively), containing 50 wt. % Diltiazem HCl. (FIG. 17). The Formulation comprising PEO/PEG at a 1:1 ratio without PAA/carrageenan, containing 50 wt. % Diltiazem HCl was prepared as a control. The results also demonstrated the drug release rate decreased with increasing the amount of mixture of PAA/carrageenan. Therefore, drug release rate can be controlled by varying the additional amount of polymer(s) having a charge opposite that of the micelle-forming drug.

### Example 12

This example illustrates the superior ability of the formulations of this invention to be gelled.

When the gelation test of the preparations comprising PAA/carrageenan/PEO/PEG at a 1:1:0:0, 1:1:1:1 or a 1:1:3:3 ratio, containing 50 wt. % Diltiazem HCl. was performed according to the method described in Test Method 2. The percent gelation of these formulations demonstrated 75.0%, 80.8% and 80.7% in JP 1st fluid, respectively.

In case of the preparation comprising PAA/PEO/PEG in a 1:9:9, the percent gelation demonstrated 78.0% and 76.9% in JP 1st fluid and JP 2nd fluid, respectively.

### Test Method 3

Pharmacokinetic Study in Beagle Dogs

Nine male beagle dogs weighing 9.3 to 13.4 kg were fasted for 18 h before administration. After oral administration of the test tablet containing 200 mg of Diltiazem HCl with 30 mL water, they were allowed free access to water, but food was withheld until the last blood sample had been taken. Blood samples were collected at 0.5, 1, 2, 3, 4, 6, 8, 10, 12, and 24 h. after administration. Subsequently, plasma was separated by centrifugation to be applied to the quantitative analysis by HPLC system with UV detection.

### Example 13

This example illustrates the influence of percent gelation of preparations on in vivo sustained drug release.

Two preparations (Preparation A; 63.4% and Preparation B; 77.6% of percent gelation in JP 1st fluid) comprising different amount of PAA/PEO/PEG, both containing 200 mg of Diltiazem HCl were used for pharmacokinetic study in beagle dogs. The results demonstrated that the Preparation B showed a sustained drug release in the lower digestive tract as well as in upper digestive tract, although Preparation A released little drug in the lower digestive tract.

To compare in vivo drug release between two preparations in detail, the area under the drug concentration in plasma curve (AUC) from 0 to 24 hr was calculated as a function of in vivo absorbed drug amount. The results demonstrated that the AUC of Preparation B (7541.2±2153.7 ng h/mL) was significantly higher than that of Preparation A (4346.1±1811.6 ng h/mL), which confirmed in vivo insufficient drug release for the preparation with lower percent gelation.

All publications, patents and patent applications mentioned in this specification are herein incorporated by reference into the specification in their entirety for all purposes. Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

What is claimed is:

1. An oral sustained release pharmaceutical formulation, said oral sustained release pharmaceutical formulation comprising:
a micelle forming drug having a charge; and
at least one polymer having an opposite charge.

2. The oral sustained release pharmaceutical formulation of claim 1, wherein said micelle forming drug is a water-soluble drug.

3. The oral sustained release pharmaceutical formulation of claim 2, wherein said a micelle forming drug has a positive charge at physiological pH.

4. The oral sustained release pharmaceutical formulation of claim 2, wherein said a micelle forming drug has a negative charge at physiological pH.

5. The oral sustained release pharmaceutical formulation of claim 2, wherein said a micelle forming drug is a basic drug.

6. The oral sustained release pharmaceutical formulation of claim 1, wherein said micelle forming drug is a member selected from the group consisting of an antidepressant, a β-adrenoceptor blocking agent, an anesthetic, an antihistamine, a phenothiazine, a tranquilizer, an antibacterial, an antibiotic, an anti-inflammatory, an analgesic, an antipyretic, and a diuretic.

7. The oral sustained release pharmaceutical formulation of claim 3, wherein said at least one polymer has a negative charge.

8. The oral sustained release pharmaceutical formulation of claim 7, wherein said at least one polymer has a carboxylic group

9. The oral sustained release pharmaceutical formulation of claim 8, wherein said at least one polymer is selected from the group consisting of polyacrylic acid, polymethacrylic acid, methylmethacrylate-methacrylic acid copolymer, carboxymethyl-cellulose, alginates, xanthan gum, gellan gum, guar gum, locust bean gum, and hyaluronic acid.

10. The oral sustained release pharmaceutical formulation of claim 9, wherein said at least one polymer is polyacrylic acid.

11. The oral sustained release pharmaceutical formulation of claim 9, wherein said other polymer has a sulfate group.

12. The oral sustained release pharmaceutical formulation of claim 11, wherein said other polymer is selected from the group consisting of carrageenan, dextran sulfate.

13. The oral sustained release pharmaceutical formulation of claim 7, the percentage gelation of the formulation is not less than approximately 70%.

14. An oral sustained release pharmaceutical formulation, said oral sustained release pharmaceutical formulation comprising:
(I) a micelle forming drug is a water-soluble basic drug having a positive charge at physiological pH,
(II) polyacrylic acid;
and further if necessary comprising
(III) a hydrogel-forming polymer substance; and
(IV) hydrophilic base

15. The oral sustained release pharmaceutical formulation of claim 14, the percentage gelation of the formulation is not less than approximately 70%.

16. The oral sustained release pharmaceutical formulation of claim 14, wherein the hydrogel-forming polymer substance is 1 or more having a viscosity-average molecular weight of 2,000,000 or higher and/or a viscosity in an aqueous 1% solution (25° C.) of 1,000 cp or higher.

17. The oral sustained release pharmaceutical formulation of claim 16, wherein the hydrogel-forming polymer substance contains at least one type of polyethylene oxide.

18. The oral sustained release pharmaceutical formulation of claim 14, wherein said the hydrophilic base is 1 or 2 or more having solubility such that the amount of water needed to dissolve 1 g base is 5 mL or less.

19. The oral sustained release pharmaceutical formulation of claim 18, wherein said the hydrophilic base is 1 or 2 or more selected from the group consisting of polyethylene glycol, sucrose, and lactulose.

20. The oral sustained release pharmaceutical formulation of claim 14, wherein further formulation comprising
(V) at least one polymer has a sulfate group.

21. The oral sustained release pharmaceutical formulation of claim 20, wherein said polymer is selected from the group consisting of carrageenan, dextran sulfate.

22. The oral sustained release pharmaceutical formulation of claim 20, the percentage gelation of the formulation is not less than approximately 70%.

23. The oral sustained release pharmaceutical formulation of claim 20, wherein the hydrogel-forming polymer substance is 1 or more having a viscosity-average molecular weight of 2,000,000 or higher and/or a viscosity in an aqueous 1% solution (25° C.) of 1,000 cp or higher.

24. The oral sustained release pharmaceutical formulation of claim 23, wherein the hydrogel-forming polymer substance contains at least one type of polyethylene oxide.

25. The oral sustained release pharmaceutical formulation of claim 20, wherein said the hydrophilic base is 1 or 2 or more having solubility such that the amount of water needed to dissolve 1 g base is 5 mL or less.

26. The oral sustained release pharmaceutical formulation of claim 25, wherein said the hydrophilic base is 1 or 2 or more selected from the group consisting of polyethylene glycol, sucrose, and lactulose.

27. The oral sustained release pharmaceutical formulation of claim 14, wherein there is approximately 10 wt % to 75 wt % of said drug, approximately 5 to approximately 50 wt % of polyacrylic acid, approximately 10 to approximately 90 wt % of hydrogel-forming polymer substance, and approximately 5 to approximately 60 wt % of hydrophilic base.

28. The oral sustained release pharmaceutical formulation of claim 20, wherein there is approximately 10 wt % to 75 wt % of said drug, approximately 5 to approximately 50 wt % of polyacrylic acid, approximately 10 to approximately 90 wt % of hydrogel-forming polymer substance, approximately 5 to approximately 60 wt % of hydrophilic base, and approximately 5 wt % to 50 wt % of polymer bearing sulfate group.

29. The oral sustained release pharmaceutical formulation of claim 4 wherein said at least one polymer has a positive charge.

30. The oral sustained release pharmaceutical formulation of claim 29 wherein said at least one polymer having a positive charge is selected from the group consisting of polyethylene imine, chitosan, polyvinylpirridinium bromide, and polydimethyl-aminoethylmethacrylate.

31. A method for extending release of a micelle forming drug, said method comprising:
orally administering a pharmaceutical formulation comprising a micelle forming drug having a charge; and at least one polymer having an opposite charge, thereby extending release of said micelle forming drug.

32. The method for extending release of claim 31, wherein said micelle forming drug is a water-soluble drug.

33. The method for extending release of claim 32, wherein said a micelle forming drug has a positive charge at physiological pH.

34. The method for extending release of claim 32, wherein said a micelle forming drug has a negative charge at physiological pH.

35. The method for extending release of claim 31, wherein said micelle forming drug is a member selected from the group consisting of an antidepressant, a β-adrenoceptor blocking agent, an anesthetic, an antihistamine, a phenothiazine, a tranquilizer, an antibacterial, an antibiotic, an anti-inflammatory, an analgesic, an antipyretic, and a diuretic.

36. The method for extending release of claim 33, wherein said at least one polymer has a negative charge.

37. The method for extending release of claim 36, wherein said at least one polymer has a carboxylic group.

38. The method for extending release of claim 37, wherein said at least one polymer is selected from the group consisting of polyacrylic acid, polymethacrylic acid, methylmethacrylate-methacrylic acid copolymer, carboxymethylcellulose, alginates, xanthan gum, gellan gum, guar gum, locust bean gum, and hyaluronic acid.

39. The method for extending release of claim 38, wherein said at least one polymer having a negative charge is polyacrylic acid.

40. The method for extending release of claim 36, wherein said at least one polymer has a sulfate group.

41. The method for extending release of claim 40, wherein said polymer is selected from the group consisting of carrageenan, dextran sulfate.

42. A method for extending release of a micelle forming drug, said method comprising: orally administering a pharmaceutical formulation comprising
(I) a micelle forming drug is a water-soluble basic drug having a positive charge at physiological pH,
(II) polyacrylic acid;
and further if necessary comprising
(III) a hydrogel-forming polymer substance; and
(IV) hydrophilic base,
thereby extending release of said micelle forming drug.

43. The method for extending release of claim 42, wherein said formulation further comprising
(V) at least one polymer has a sulfate group, thereby extending release of said micelle forming drug.
